(19) United States
(12) Patent Application Publication
(10) Pub. No.: US 2018/0149718 A1
(43) Pub. Date: May 31, 2018

(54) WEARABLE SHOULDER COIL

(71) Applicant: Shanghai Neusoft Medical Technology Co., Ltd., Shanghai (CN)

(72) Inventors: Jian GUO, Shanghai (CN); Guanjun MA, Shanghai (CN)

(21) Appl. No.: 15/825,000

(22) Filed: Nov. 28, 2017

(30) Foreign Application Priority Data
Nov. 29, 2016 (CN) 201611076383.6

(51) Int. Cl. G01R 33/3415; A61B 5/055; G01R 33/36

(52) U.S. Cl. CPC G01R 33/3415; A61B 5/6813; G01R 33/36; A61B 5/055

(57) ABSTRACT

Provided is a wearable shoulder coil including a signal receiving device and a signal transmitting device. The signal receiving device includes a flexible coil array that forms an examination space for accommodating an examined region. The signal transmitting device is provided with a processing unit to process signals, and is connected with the signal receiving device. The coil array may receive a first signal and transmit the first signal to the processing unit. The signal transmitting device may generate a second signal from the first signal and output the second signal.

### CROSS REFERENCE TO RELATED APPLICATION

This present application claims priority to Chinese Patent Application No. 201611076383.6, entitled “WEARABLE SHOULDER COIL” filed on Nov. 29, 2016, which are hereby incorporated by reference for all purposes.

### BACKGROUND

The present disclosure relates to a wearable shoulder coil.

Among a number of medical imaging examination methods, magnetic resonance imaging has advantages of good soft tissue resolution and capability of imaging at any angle, making it one of the important methods for diagnosing shoulder joint diseases. A shoulder joint is the most flexible ball and socket joint of the whole body, and may move in a large range. Moreover, anatomical locations of patients with shoulder joint diseases are widely different and may affect quality of images which are reconstructed from CT scanning for shoulder joint.

NEUSOFT MEDICAL SYSTEMS CO., LTD. (NMS), founded in 1998 with its world headquarters in China, is a leading supplier of medical equipment, medical IT solutions, and healthcare services. NMS supplies medical equipment with a wide portfolio, including CT, Magnetic Resonance Imaging (MRI), digital X-ray machine, ultrasound, Positron Emission Tomography (PET), Linear Accelerator (LINAC), and biochemistry analyser. Currently, NMS' products are exported to over 60 countries and regions around the globe, serving more than 5,000 renowned customers. NMS's latest successful developments, such as 128 Multi-Slice CT Scanner System, Superconducting MRI, LINAC, and PET products, have led China to become a global high-end medical equipment producer. As an integrated supplier with extensive experience in large medical equipment, NMS has been committed to the study of avoiding secondary potential harm caused by excessive X-ray irradiation to the subject during the CT scanning process.

### BRIEF DESCRIPTION OF DRAWINGS

FIG. 1 illustrates a structural schematic diagram of a wearable shoulder coil according to an example of the present disclosure.

FIG. 2 illustrates a structural schematic diagram of the wearable shoulder coil in FIG. 1 at another angle.

FIG. 3 illustrates a structural schematic diagram of a coil array according to an example of the present disclosure.

FIG. 4 illustrates a structural schematic diagram of a signal transmitting device according to an example of the present disclosure.

### DETAILED DESCRIPTION

Illustrative embodiments will be described in detail herein, examples of which are shown in the accompanying drawings. When the following description refers to the accompanying drawings, the same numerals in the different drawings denote the same or similar elements unless otherwise indicated. The examples described in the following embodiments are not representative of all examples coincident with the present disclosure. In contrast, they are merely examples of devices and methods coincident with some aspects of the present disclosure as detailed in the appended claims.

The terminology used in the present disclosure is for the purpose of describing particular examples only, and is not intended to be limiting of the present disclosure. The singular forms such as “a”, ‘said”, and “the” used in the present disclosure and the appended claims are also intended to include multiple, unless the context clearly indicates otherwise. It is also to be understood that the term “and/or” as used herein refers to and includes any or all possible combinations of one or more associated listed items.

It is to be understood that although different information may be described using the terms such as first, second, third, etc. in the present disclosure, these information should not be limited to these terms. These terms are used only to distinguish the same type of information from each other. For example, the first information may also be referred to as the second information without departing from the scope of the present disclosure, and similarly, the second information may also be referred to as the first information. Depending on the context, the word such as “if” used herein may be interpreted as “when” or “as” or “determining in response to”.

Shoulder coils may be designed in two forms: a combination of a partially flexible and partially hard structure, or an entirely hard structure. For a shoulder coil with a partially flexible and partially hard structure, upper and lower parts of the shoulder coil may be designed to be flexible, while channels and amplifier circuits at a side of the shoulder coil may be designed to be protected by a hard shell. With an increase in a number of coil channels, a large number of components and amplifier circuits are provided so that an area of the hard shell may not be reduced. Therefore, the shoulder coil may be poor in user comfort. Besides, a strip of cloth, such as a bandage, is needed to bind the shoulder coil to a shoulder of a patient. A shape and/or size of a shoulder coil with an entirely hard structure cannot be adjusted, and therefore cannot fit patients of different shapes. To scan different patients of different shapes, a plurality of shoulder coils of different sizes are needed. A fixed shape of the shoulder coil may result in inconvenience when fixing the shoulder coil on a body of a patient. A mat may be used to fill in a gap between the shoulder coil and a shoulder of the patient such that the shoulder of the patient stays still with respect to the shoulder coil during the examination. Therefore, user experience may be bad.

The present disclosure provides a wearable shoulder coil. As shown in FIGS. 1 to 3, a wearable shoulder coil shown according to an example may include a signal receiving device 10 and a signal transmitting device 20. The signal receiving device 10 may include a hard shell 12 as well as a flexible coil array 11 and a flexible body 13 that are configured on the hard shell 12. The hard shell 12 may be made of a rigid material to have a set shape. The signal receiving device 10 may be bound on the body of a user, for example, bound to a shoulder joint of the user. The coil array 11 may be sealed in the flexible body 13. The coil array 11 and the flexible body 13 may be flexible in structure so that they may be well fitted to a body surface of the user. Herein the flexible material may comprise Ethylene Vinyl Acetate (EVA) foam. The coil array 11 may extend outwards from both sides of the hard shell 12 to form a U-shaped examination space 17 corresponding to an examined region of the user. In this way, the coil array 11 may cover a shoulder joint (humeral head), pectoralis major muscle, clavicle, and scapula to comprehensively detect and receive magnetic resonance signals generated by the shoulder joint part. For example, a magnetic resonance system may generate a uniform magnetic field in the examined region, and the coil array 11 may receive radio frequency (RF) signals at a high signal-to-noise ratio. The signal transmitting device 20 may include a processing unit 22 which may process the received RF signals. Control circuits such as an amplifier circuit, a driving circuit, and an identification circuit may be provided in the processing unit 22 to process the signals collected by the signal receiving device 10. The signal transmitting device 20 may be connected with the signal receiving device 10. The signals may be transmitted to the processing unit 22 to be processed and processed signals may be output through the signal transmitting device 20.

The signal receiving device 10 and the signal transmitting device 20 are designed to be separate, where the coil array 11 of the signal receiving device 10 may only retain a detecting unit that receives body signals. Other signal processing circuits may be moved to the outside of the signal receiving device 10, for example, to the processing unit 22 of the signal transmitting device 20. A connector socket may be provided on the hard shell 12 of the signal receiving device 10. Moreover, the hard shell 12 and the signal transmitting device 20 are designed into an integrated module to reduce the space of a connecting module. The configuration that the coil array 11 of the signal receiving device 10 retains only the detecting unit and other signal processing circuits are moved to the outside of the signal receiving device 10 may enable the area of the hard shell 12 on the signal receiving device 10 to be reduced so as to ensure that flexibility of only a small part of the coil array corresponding to the hard shell are affected and most part of the coil array 11 remains a flexible structure. The signal receiving device 10 may flexibly cover the examined region of a user, improving wearing comfort. Since it may be applied to users of different shapes, applicability may be also improved.

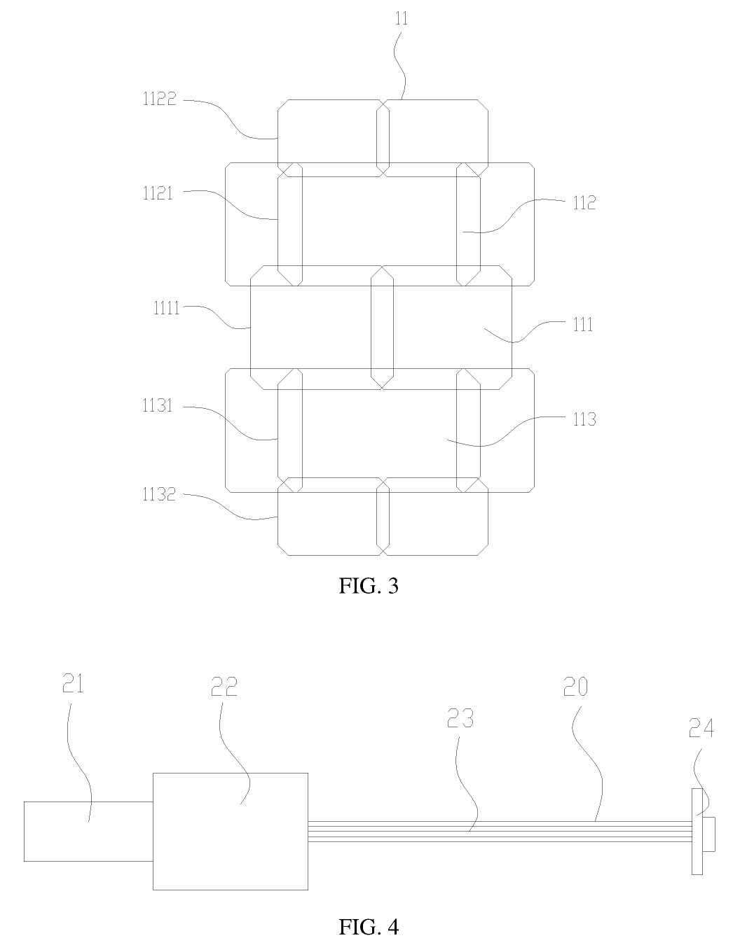

As shown in FIG. 3, the coil array 11 may include a first unit 111, a second unit 112 and a third unit 113 provided on separate sides of the first unit 111, respectively. The hard shell 12 may be located right above the first unit 111. The second unit 112 and the third unit 113 may output signals to the hard shell 12, respectively. The coil array 11 may be divided into upper and lower parts. The second unit 112 and the third unit 113 may mainly cover a shoulder joint (humeral head), a pectoralis major muscle, a clavicle, and a scapula when the wearable shoulder coil is bound to the examined region of the user. The first unit 111 may be in the middle of the coil array 11 and may mainly cover a side of a humerus. The second unit 112 and the third unit 113 may surround the first unit 111, increasing a scanning field of view of the shoulder coil.

The second unit 112 and the third unit 113 may be arranged symmetrically, so that a wearable shoulder coil is compatible with both a left and a right shoulder of the user for detection, which may reduce a number of accessories and increases flexibility and convenience of use.

The first unit 111 may include one or more first RF coils 1111. Each of the first RF coils 1111 of the first unit 111 may partially intersect with one or more adjacent first RF coils 1111. Herein, the coils intersecting one another means that the coils overlap one another. Two adjacent RF coils may be arranged to partially overlap so as to allow a sum of the magnetic fluxes passing through the overlapped portion to be zero. In this way, a coupling between the RF coils may be eliminated and electromagnetic interference between coils may be reduced. In an example, a number of the first RF coils 1111 may be set to two and the two first RF coils may partially intersect. By arranging two first RF coils 1111, an area of imaging may be enlarged and a signal-to-noise ratio of the first unit 111 may be increased at the same time so that acquired image signals may be good in quality.

The second unit 112 may include one or more second RF coils. When the second unit 112 includes a plurality of second RF coils, each of the second RF coils of the second unit 112 may partially intersect with one or more adjacent second RF coils. In addition, as shown in FIG. 3, the first unit 111 and the second unit 112 may partially intersect. Two adjacent second RF coils in the second unit 112 may be arranged to partially overlap so as to allow a sum of magnetic fluxes passing through the overlapped portion to be zero. In this way, mutual inductance between the RF coils may be zero and electromagnetic interference between RF coils may be reduced. At the same time, the partial intersection of the first unit 111 and the second unit 112 may decouple the first unit 111 and the second unit 112, reducing the electromagnetic interference therebetween and improving the quality of the imaging signal.

As further shown in FIG. 3, the second RF coils may include a second primary RF coil 1121 and at least one second auxiliary RF coil 1122. The second primary RF coil 1121 and the at least one second auxiliary RF coil 1122 may partially intersect with the first unit 111. The at least one second auxiliary RF coils 1122 may partially intersect with the second primary RF coil 1121. The adjacent second auxiliary RF coils 1122 may partially intersect with each other. In a particular example, the second primary RF coil 1121 may be arranged in the middle, four second auxiliary RF coils 1122 may be arranged around the periphery of the second primary RF coil 1121, where the four second auxiliary RF coils 1122 are all partially intersected with the second primary RF coil 1121, and the adjacent second auxiliary RF coils 1122 are partially intersected with each other. The second primary RF coil 1121 may partially intersect with the first RF coil 1111 of the first unit 111 and a part of the second auxiliary RF coils 1122 adjacent to the first RF coil 1111 may partially intersect with the first RF coil 1111. The second RF coils are subdivided into the second primary RF coil 1121 and at least one second auxiliary RF coil 1122 so that a size and an orientation of the second RF coils may be set according to an area to be covered by the second RF coils to improve a quality of the imaging signals of the second unit 112.

The area of the second primary RF coil 1121 may be larger than an area of any of the second auxiliary RF coils 1122, that is, the area covered by the second primary RF coil 1121 is slightly larger than the area covered by any of the second auxiliary RF coils 1122 so that a better signal penetration may be realized, and the imaging quality of the shoulder joint area may be improved. The second primary RF coil 1121 may mainly cover and detect the pectoralis major muscle, the clavicle and/or the scapula. A large second primary RF coil 1121 may have a good signal penetrating effect.

As further shown in FIG. 3, the third unit 113 may include one or more third RF coils. Each of the third RF coils of the third unit 113 may partially intersect with one or more adjacent third RF coils. In addition, the third unit 113 and the first unit 111 may partially intersect with each other. The adjacent third RF coils in the third unit 113 are partially overlapped to reduce the electromagnetic interference between the coils, thereby achieving decoupling between adjacent RF coils. At the same time, the first unit 111 and the third unit 113 are partially intersected with each other, decoupling between the first unit 111 and the third unit 113, reducing the electromagnetic interference between the first unit 111 and the third unit 113, and improving the quality of the imaging signals.

The third RF coil may include a third primary RF coil 1131 and at least one third auxiliary RF coil 1132. The third primary RF coil 1131 and the at least one third auxiliary RF coil 1132 may partially intersect with the first unit 111. The at least one third auxiliary RF coil 1132 may partially intersect with the third primary RF coil 1131. The adjacent third auxiliary RF coils 1132 may partially intersect with each other. In a particular example, the third primary RF coil 1131 may be arranged in the middle, and four third auxiliary RF coils 1132 may surround the periphery of the third primary RF coil 1131, where the four third auxiliary RF coils 1132 are all partially intersected with the third primary RF coil 1131, and the adjacent third auxiliary RF coils 1132 are partially intersected with each other. The third primary RF coil 1131 may partially intersect with the first RF coil 1111 of the first unit 111 and a part of the third auxiliary RF coils 1132 adjacent to the first RF coil 1111 may partially intersect with the first RF coil 1111. The third RF coil may be subdivided into the third primary RF coil 1131 and at least one third auxiliary RF coil 1132 so that a size and an orientation of the third RF coil may be set according to an area to be covered by the third RF coil to improve the quality of the imaging signal of the third unit 113.

The area of the third primary RF coil 1131 may be larger than the area of any of the third auxiliary RF coils 1132. Herein the area covered by the third primary RF coil 1131 is slightly larger than that of any of the third auxiliary RF coils 1132 so that a better signal penetration may be realized, and the imaging quality of the shoulder joint area may be improved. The third unit 113 and the second unit 112 are provided on both sides of a shoulder joint, respectively. The third primary RF coil 1131 and the second primary RF coil 1121 cover the pectoralis major muscle, the clavicle and/or the scapula corresponding to both sides of the shoulder joint, respectively. The third primary RF coil 1131 may cover a majority of the area of the region and other third auxiliary RF coils 1132 may then extend an imaging range to improve a signal-to-noise ratio while ensuring the penetration, thereby stabilizing the imaging signal and ensuring good signal quality.

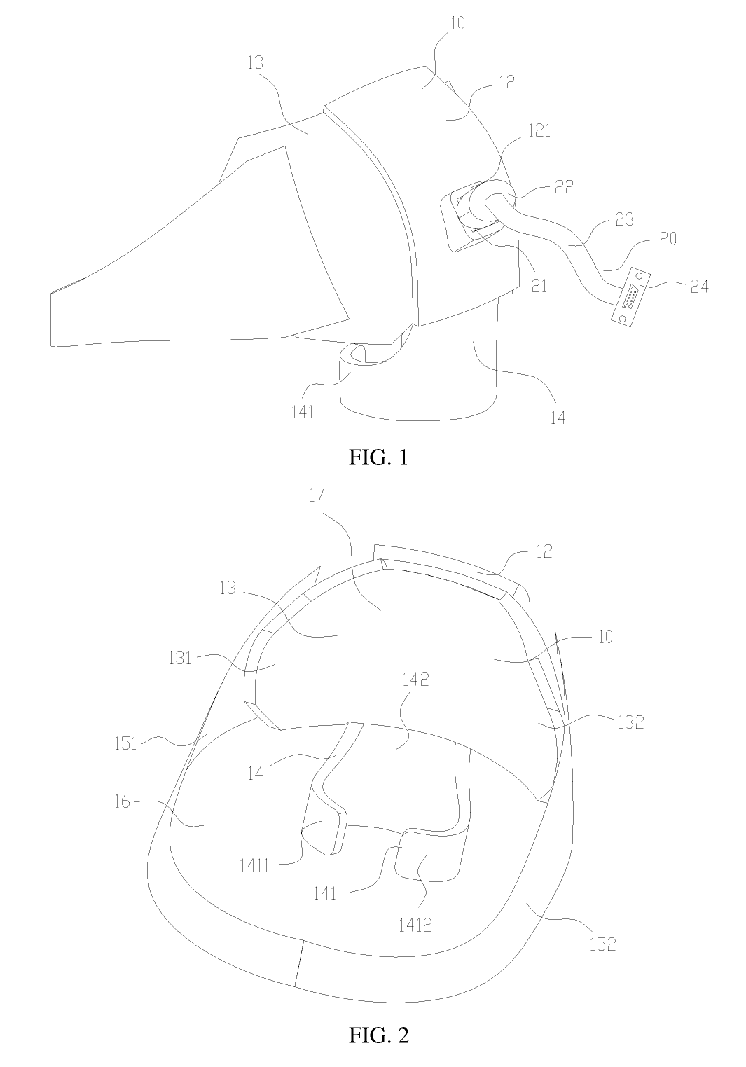

As shown in FIGS. 1 and 2, the hard shell 12 may be provided with a first connection portion 121 and one end of the signal transmitting device 20 may be provided with a second connection portion 21 mating with the first connection portion 121. One of the first connection portion 121 and the second connection portion 21 may be formed into a plug shape, and the other may be formed into a socket shape. The coil array 11 may output the received signals to the first connection portion 121 and the signal receiving device 10 may be connected to the signal transmitting device 20 through the first connection portion 121. The hard shell 12 may secure the coil array 11 with an effect of supporting and positioning. The first connection portion 121 is provided on the hard shell 12 with its position being relatively fixed so that the connection with the signal transmitting device 20 may be easy and convenient. The first connection portion 121 and the second connection portion 21 are arranged in such a manner that the signal transmitting device 20 and the signal receiving device 10 may be quickly connected and disconnected, facilitating transmission of signals and connection. Signals collected by the coil array 11 may be transmitted to the processing unit 22 via the second connection portion 21 connected to the hard shell 12 after the signal transmitting device 20 and the signal receiving device 10 are connected. Accordingly, after receiving the signals, the processing unit 22 may process the signals and transmit the processed signals to a system device.

As shown in FIGS. 1 and 4, the signal transmitting device 20 may further include a cable portion 23, one end of which is provided with the second connection portion 21, and the other end is provided with a plug portion 24, where the plug portion is configured to connect with the system device. The signal receiving device 10 may be quickly connected to the system device through the signal transmitting device 20 in a coupling manner. The processing unit 22 may be located between the second connection portion 21 and the plug portion 24 and may be close to the second connection portion 21. Close arrangement of the processing unit 22 to the second connection portion 21 may facilitate the processing unit to quickly receive image signals transmitted from the first connection portion 121. Moreover, the smaller a transmission distance of signals on the cable portion 23 is, the less the signal loss is. After the processing unit 22 identifies and amplifies valid image signals, the processed signals may be transmitted to the system device. In this way, the signals received by the system device may be high in quality and good in stability.

As shown in FIGS. 1 and 2, the flexible body 13 may include a first connecting end 131 and a second connecting end 132. The first connecting end 131 and the second connecting end 132 may be provided with or connected with a first connecting member 151 and a second connecting member 152, respectively. Separate ends of the first and second connecting members 151 and 152 may be connected to form an accommodating space 16 with the coil array 11. An examined region of a user may be located in the accommodating space 16. The flexible body 13 may enclose the coil array 11. The flexible body 13 may be connected to the hard shell 12 and extend along both sides of the hard shell 12. The first connecting end 131 and the second connecting end 132 are provided on the flexible body 13. The coil array 11 may extend outwards from both sides of the hard shell 12 to form a U-shaped examination space 17. The first connecting member 151 and the second connecting member 152 may be made of a flexible material such as a bandage and may tighten the first connecting end 131 and the second connecting end 132 so that the flexible body 13 is fitted to the skin of a user. In addition, the first connecting member 151 and the second connecting member 152 may be connected with each other in a manner of knotting, a buckle, a Velcro, etc., so that the signal receiving device 10 may be conveniently fixed to the examined region of a user.

The hard shell 12 may be provided with a fixing member 14. The fixing member 14 may comprise a connecting portion 141, and the connecting portion 141 may form a fixing space 142 for fixing a position of the hard shell 12. The hard shell 12 and the flexible body 13 may be bound to the examined region of a user through the first connecting member 151 and the second connecting member 152. The inclusion of fixing member 14 in the hard shell 12 may secure the mounting position of the hard shell 12. Herein the connecting portion 141 may be fixed to a particular region of the user so as to fix the position the hard shell 12. For example, the connecting portion 141 may be secured to an arm of the user in the form of a resilient ring with elasticity or a Velcro or by a combination of a snap and an elastic band.

With continued reference to FIGS. 1 and 2, in a particular example, the connecting portion 141 may include a first fixing end 1411 and a second fixing end 1412. The first fixing end 1411, the second fixing end 1412, and connecting point of the fixing member 14 and the hard shell 12 may be triangularly distributed. The first fixing end 1411 may be connected with the second fixing end 1412 in a joining manner to form an annular fixing space 142. The connecting portion 141 may be a flexible thin-walled member. The first fixing end 1411 and the second fixing end 1412 may be connected through ends to form an annular fixing space 142. The connecting portion 141 is fixed to a user so that an arm of the user may pass through the fixing space 142 and be fixed. The triangular distribution of the first fixing end 1411, the second fixing end 1412, and the connecting point of the fixing member 14 and the hard shell 12 may allow a space enclosed by the first fixing end 1411, the second fixing end 1412 and the hard shell 12 to be adjustable so as to facilitate the hard shell 12 to be fitted to the shoulder.

For simplicity and illustrative purposes, the present disclosure is described by referring mainly to examples thereof without intending to limit the present disclosure. In the above descriptions, numerous specific details are set forth to provide a thorough understanding of the present disclosure. It will be readily apparent however, that the present disclosure may be practiced without limitation to these specific details. In other instances, some methods and structures have not been described in detail so as not to unnecessarily obscure the present disclosure. As used herein, the terms “a” and “an” are intended to denote at least one of a particular element, the term “includes” means includes but not limited to, the term “including” means including but not limited to, and the term “based on” means based at least in part on.

Therefore, those skilled in the art, without departing from the scope of the technical scheme of the present disclosure, based on technical essences of the present disclosure, may make a plurality of changes and modifications of the technical scheme of the present disclosure by the method and technical content disclosed above. Thus, any simple alterations, equal changes and modifications should fall within the protection scope of the technical scheme of the present disclosure. Accordingly, other embodiments are within the scope of the following claims.

1. A wearable shoulder coil, comprising:
a signal receiving device comprising a flexible coil array to receive a first signal; and
a signal transmitting device coupled with the signal receiving device and provided with a processing unit for processing the first signal,
wherein the coil array transmits the received first signal to the processing unit, and
wherein the signal transmitting device is configured to generate a second signal from the first signal by the processing unit and output the second signal.

2. The wearable shoulder coil according to claim 1, wherein
the signal receiving device comprises a hard shell along which the coil array extends outwards and forms an examination space for accommodating an examined region, and
the signal transmitting device is coupled to the hard shell.

3. The wearable shoulder coil according to claim 2, wherein
the hard shell is provided with a first connection portion,
one end of the signal transmitting device is provided with a second connection portion mating with the first connection portion, and
the first signal collected by the coil array is input to the first connection portion and transmitted to the processing unit via the second connection portion connected to the hard shell.

4. The wearable shoulder coil according to claim 3, wherein
the signal transmitting device further comprises a cable portion,
one end of the cable portion is provided with the second connection portion, and the other end of the cable portion is provided with a plug portion, and
the processing unit is close to the end on which the second connection portion is provided.

5. The wearable shoulder coil according to claim 2, wherein
the hard shell is provided with a fixing member,
the fixing member is provided with a connecting portion, and
the connecting portion is provided with a fixing space for fixing the hard shell.

6. The wearable shoulder coil according to claim 5, wherein
the connecting portion comprises a first fixing end and a second fixing end,
the first fixing end, the second fixing end, and the connecting point of the fixing member and the hard shell are triangularly distributed, and
the first fixing end is connected with the second fixing end in a joining manner to form the annular fixing space.

7. The wearable shoulder coil according to claim 2, wherein the coil array comprises:
a first unit above which the hard shell is located; and
a second unit and a third unit arranged on opposite sides of the first unit, wherein the second unit and the third unit respectively output a signal to the hard shell.

8. The wearable shoulder coil according to claim 7, wherein the second unit and the third unit are symmetrically arranged.

9. The wearable shoulder coil according to claim 7, wherein the first unit comprises one or more first radio frequency (RF) coils.

10. The wearable shoulder coil according to claim 9, wherein every adjacent two of the first RF coils are partially intersected with each other.

11. The wearable shoulder coil according to claim 7, wherein the second unit comprises one or more second RF coils.

12. The wearable shoulder coil according to claim 11, wherein
every adjacent two of the second RF coils are partially intersected with each other, and
the first unit and the second unit are partially intersected with each other.

13. The wearable shoulder coil according to claim 12, wherein the second RF coils comprises:
a second primary RF coil partially intersected with the first unit; and
one or more second auxiliary RF coils which are partially intersected with the first unit, respectively, and are partially intersected with the second primary RF coil, respectively,
wherein adjacent two of the second auxiliary RF coils are partially intersected with each other.

14. The wearable shoulder coil according to claim 13, wherein an area of the second primary RF coil is larger than an area of any of the second auxiliary RF coils.

15. The wearable shoulder coil according to claim 7, wherein the third unit comprises one or more third RF coils.

16. The wearable shoulder coil according to claim 15, wherein
every adjacent two of the third RF coils are partially intersected with each other, and
the third unit and the first unit are partially intersected with each other.

17. The wearable shoulder coil according to claim 16, wherein the third RF coil comprises:
a third primary RF coil partially intersected with the first unit; and
one or more third auxiliary RF coils which are partially intersected with the first unit, respectively, and are partially intersected with the third primary RF coil, respectively,
wherein every adjacent two of the third auxiliary RF coils are partially intersected with each other.

18. The wearable shoulder coil according to claim 17, wherein an area of the third primary RF coil is larger than an area of any of the third auxiliary RF coils.

19. The wearable shoulder coil according to claim 1, wherein
the signal receiving device further comprises a flexible body, and
the coil array is sealed in the flexible body.

20. The wearable shoulder coil according to claim 19, wherein the flexible body comprises:
a first connecting end provided with a first connecting member; and
a second connecting end provided with a second connecting member,
wherein the first connecting member and the second connecting member are configured to be connected with each other by connecting the first connecting end and the second connecting end to form an accommodating space with the coil array.
